(19) United States
(12) Patent Application Publication
(10) Pub. No.: US 2007/0219129 A1
(43) Pub. Date: Sep. 20, 2007

(54) hsp90 decoy peptides and uses thereof

(76) Inventors: Kirkwood A. Pritchard JR., Elm Grove, WI (US); Hao Xu, Wauwatosa, WI (US); Yang Shi, Wauwatosa, WI (US)

Correspondence Address: QUARLES & BRADY LLP, 411 E. WISCONSIN AVENUE, SUITE 2040, MILWAUKEE, WI, 53202-4497, US

(21) Appl. No.: 11/505,036

(22) Filed: Aug. 16, 2006

Related U.S. Application Data
(60) Provisional application No. 60/708,919, filed on Aug. 17, 2005.

(51) Int. Cl. A61K 38/17

(52) U.S. Cl. 514/12

(57) ABSTRACT

A method of inhibiting hsp90 association with eNOS in a patient, comprising the step of treating a patient with an effective amount of a pharmaceutical composition comprising an hsp90 decoy peptide.

### CROSS-REFERENCE TO RELATED APPLICATIONS

This application claims the benefit of U.S. provisional application 60/708,919, filed Aug. 17, 2005 and incorporated by reference herein.

### STATEMENT OF FEDERALLY SPONSORED RESEARCH OR DEVELOPMENT

This invention was made with United States government support awarded by the following agency: NIH Grant No. 5R01HL071214. The United States government has certain rights in this invention.

### BACKGROUND OF THE INVENTION

Previous studies showed that the association of heat shock protein 90 (hsp90) with endothelial nitric oxide synthase (eNOS) played an important role in the generation of nitric oxide (.NO).¹ Previous studies from our laboratory revealed that inhibition of hsp90 ATPase-dependent chaperone activity not only decreased stimulated .NO generation but also increased eNOS-dependent superoxide anion (O₂.⁻) production.²⁻⁴ These reports suggested that inhibiting hsp90-dependent signaling with eNOS allows eNOS to generate O₂.⁻ upon stimulation rather than .NO. As .NO plays such a central role in vascular biology such changes in enzyme function will likely have a major impact on physiology and angiogenesis.

An earlier report by Sessa and associates showed, using a yeast two-hybrid system, that hsp90 interacted with eNOS at 290-400 amino acids (aa) of eNOS.⁵ Previous studies by Pagano and associates showed that small peptides corresponding to a portion of gp91phox could act as a decoy peptide to inhibit assembly of vascular NADPH oxidoreductase and therefore vascular O₂.⁻ generation.⁶

Identification of novel heat shock protein 90 decoy peptides and disclosure of use of these peptides in specific therapeutic applications is needed in the art of therapeutic interventions.

### BRIEF SUMMARY OF THE INVENTION

On the basis of the studies described above, we reasoned that if the binding domain of eNOS and hsp90 could be better resolved, then the primary amino acid sequences derived from eNOS might be effective inhibitors of hsp90 association with eNOS. The Examples below disclose our experiments designed to determine where hsp90 bound to eNOS and develop a novel decoy peptide that would disrupt hsp90 interactions with eNOS. In this application, we report 1) the location where hsp90 binds to eNOS and 2) that an eNOS-derived decoy peptide is a potent inhibitor of stimulated .NO production, vasodilation and growth of B16 melanoma tumors in mice.

In one embodiment, the present invention is a pharmaceutical composition comprising an hsp90 decoy peptide, preferably wherein the peptide comprises SB2 (SEQ ID NO:5). Preferably, the peptide additionally comprises a protein translocation domain.

In one embodiment of the invention the peptide is TSB2 (SEQ ID NO:6).

In another embodiment, the present invention is a method of inhibiting hsp90 association with eNOS in a patient, comprising the step of treating a patient with an effective amount of the pharmaceutical composition described above.

Other features, objects and embodiments of the present invention will be apparent to one of skill in the art after review of the specification, claims and drawings.

### BRIEF DESCRIPTION OF THE SEVERAL VIEWS OF THE DRAWINGS

The file of this patent contains at least one drawing executed in color. Copies of this patent with color drawing(s) will be provided by the Patent and Trademark Office upon request and payment of the necessary fee.

FIG. 1. eNOS-derived peptides decrease hsp90 association with eNOS. Western blot anlaysis of immunoprecipitates of eNOS from a pooled lysate from proliferating Bovine Aortic Endothelial Cells (BAEC) show that B1 (290-310 aa), B2 (301-320 aa) and B3 (310-330 aa) decreased hsp90 association with eNOS. This experiment was repeated at least 5 times with lysates from at least 6 different lysate preparations. (p<0.05, n=5).

FIG. 2. B2 inhibits BAEC-dependent basal and stimulated .NO Generation: Treatment of BAEC cultures with B2 and PEP1 (21-residue peptide carrier, KETWWETWWTEWSQPKKKRKV, SEQ ID NO:9, Morris et al., Nature Biotech, 19:1173-1176, 2001) markedly inhibit basal and stimulated .NO (measured as nitrite+nitrate by ozone chemiluminescence) production and inhibit the association of hsp90 with eNOS in the treated BAEC. PEP1-treated and untreated BAEC cultures produce high levels of .NO and demonstrate high levels of hsp90 association with eNOS. BAEC cultures treated with B2:PEP1 (1:20, 5 nM B2, final concentration) generate low levels of .NO and demonstrate low levels of hsp90 association compared to untreated controls or PEP 1-treated cultures.

FIG. 3. TSB2, an eNOS-derived decoy peptide, inhibits ACh-induced vasodilation of facialis arteries. The B2 was reduced further based on the amino acids it had in common with B1 and B3 and where the amino acids were located on the 3D-structure of eNOS. A TAT protein transduction domain was added to the shortened form of B2 at the N-terminal end of the peptide to yield a new peptide called TSB2. Acute exposure (10 min) of isolated, pressurized murine facialis arteries to TSB2 (10 μg/mL) markedly reduces ACh-dependent vasodilation compared to untreated vessels. (p<0.02, n=6-10)

FIG. 4. TSB2 inhibits ACh-dependent vasodilation offacialis arteries ex vivo. C57BL/6 mice were treated with TSB2 (1 mg/kg) or PBS (100 μL) for 2 weeks. At the end of this treatment period, facialis arteries from TSB2-treated and untreated C57BL/6 mice were isolated, pressurized, and examined for responses to ACh as previously described.⁹ This line graph shows that TSB2 treatments alter vascular responses to ACh. Not only is ACh-induced vasodilation reduced but other mechanisms of vasodilation are developing in vessels from TSB2-treated mice based on L-Nitro Arginine Methyl Ester (L-NAME), a specific inhibitor of NOS enzymes, failing to reduce vasodilation to baseline.

FIG. 5. TSB2 uncouples eNOS activity in native EC on the aortas of C57BL/6 mice. This figure shows confocal microscopy images of fluorescent intensity of aortas perfused with MOPS buffer (2 mL/min) containing hydroethidine (10 μM) without and with L-NAME (200 μM) as described in Methods. TSB2 markedly increases hydroethidine staining in the nuclei of vascular EC on aortas of C57BL/6 mice (upper right) compared to the levels of hydroethidine staining in control aortas perfused with MOPS buffer containing hydroethidine alone (upper left). Hydroethidine staining decreases in native EC on aortas perfused with MOPS buffer containing hydroethidine, TSB2 and L-NAME (lower right) compared to the levels of hydroethidine staining in aortas perfused with MOPS buffer containing hydroethidine and TSB2 (upper right) or control aortas perfused MOPS containing hydroethidine alone (upper left).

FIG. 6. Differential effects of TSB2 on B16 melanoma cells, EC and K562 leukemia cells: TSB2 has no significant effects on the viability of B16 melanoma cells as measured by changes in LDH release (A) but does decrease proliferation of EC by 58% and K562 cells by 28% (B).

FIG. 7. TSB2 inhibits the growth of B16 melanoma tumors in mice: These figures show the growth of B16 melanoma tumors in mice treated with either PBS, or TSB2 at 1× and 3× doses as described in methods. TSB2 markedly reduces the growth of B16 melanoma tumors in C57BL/6 mice during the experiment (A). Such slow rates of growth are confirmed in weight (B) and volume (C) measurements made on tumors isolated from the mice at the end of the experiment.

FIG. 8. TSB2 increases production of angiostatin in vivo: Immunofluorescent studies reveal TSB2 induces a marked increase in angiostatin expression (bright green) in the hearts of C57BL/6 mice. The blue dots are nuclei of myocytes stained with ToPro-3.

### DETAILED DESCRIPTION OF THE INVENTION

In General.

Heat shock protein 90 (hsp90) binds to eNOS to increase nitric oxide (.NO) generation. Disruption of this interaction allows eNOS to generate superoxide anion (O₂.⁻) upon activation. The present study demonstrates that generation of overlapping peptides based on eNOS sequences from 291 to 300 aa and incubation with cell lysates from proliferating BAEC reveal that hsp90 associates with eNOS at amino acids (aa) 301-320. Treating bovine aortic endothelial cells with the 301-320 peptide (B2) decreases stimulated .NO production and hsp90 association.

Redesign of the B2 peptide to include a protein transduction domain (PTD) and shortening the peptide to 15 aa resulted in a new decoy peptide that impairs vasodilation in vitro and in vivo, uncouples eNOS activity to increase eNOS-dependent O₂.⁻ generation in native EC on mouse aortas, inhibits proliferation of EC and K562 cells but not B16 melanoma cells. Chronic TSB2 treatments of mice inoculated with B16 melanoma dramatically impairs tumor growth with respect to weight and volume. Immunofluorescence studies indicate TSB2 increases angiostatin production in vascular tissues. Taken together, these data demonstrate that an eNOS-derived decoy peptide effectively impairs vascular EC .NO generation, vasodilation and growth of B16 melanoma cells in mice.

hs90 Decoy Peptides of the Present Invention

We disclose a category of peptide derived from the eNOS sequence that we call “hsp90 decoy peptides”. These peptides uncouple eNOS activity to increase eNOS dependent O₂.⁻ generation in native EC. These peptides impair vasodialation in vitro and in vivo as evidenced by ex vivo studies.

In one embodiment of the invention, the hsp90 decoy peptide comprises peptides B1, B2 or B3 (see Table 1), preferably connected with a protein transduction system. Most preferably, Applicants have demonstrated TSB2 (SEQ ID NO:6) wherein TAT is operably connected to peptide B2 with a linking residue. In TSB2, this linking residue is an alanine residue (A). However, one may easily substitute other residues as a linker. For example, one may replace alanine with glycine.

In another embodiment of the present invention, the hsp90 decoy peptide is a peptide comprising SB2 (SEQ ID NO:3). The hsp90 decoy peptide comprising SB2 may additionally comprise residues (preferably no more than 7 to 19 residues, most preferably 1, 2 or 3 residues) at each end of the peptide. Preferably, the entire peptide will be between 22 and 34 residues. Preferably, these additional residues are the flanking residues found in either bovine or human endothelial nitric oxide synthase. Preferably, the hsp90 decoy peptide includes a protein transduction system.

One of skill in the art would understand that any of the peptides described above would have identical activity if substituted with conservative amino acid substitutions. For example, any or all of the following amino acid substitutions may be made: D for E, E for D, I for L, L for I, V for L, L for V, I for V, V for I, S for T and T for S. Applicants mean to include these conservative amino acid substitutions in their definition of hsp90 decoy peptides and B1, B2, etc.

Preferably, the peptide is coupled to a protein transduction domain, most preferably TAT. Other suitable protein transduction domains would include NGR (Fibronectin-like domain 3, J Cell. Biol., 139[6]:1567-1581, 1997) and antennapedia transduction domain.

TABLE 1
hsp90 Decoy Peptides

Bovine Endothelial Nitric Oxide Synthase amino acid sequence (291-330)

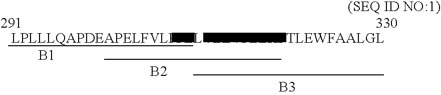

The shaded region in SEQ ID NO:1 is identical between the human eNOS
sequence and the bovine eNOS sequence.

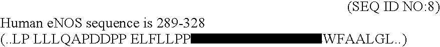

| | |
|---|---|
| B1 (291-311) | (SEQ ID NO:2) |

LPLLLQAPDEAPELFVLPPE

| | |
|---|---|
| B2 (301-320) | (SEQ ID NO:3) |

APELFVLPPELVLEVPLEHP

| | |
|---|---|
| B3 (311-330) | (SEQ ID NO:4) |
| LVLEVPLEHPTLEWFAALGL | |
| SB2 (300-313) | (SEQ ID NO:5) |

ELVLEVPLEHPTLE

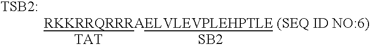

Please note that the non-underlined residue in TSB2 is a linker between
TAT and SB2 that we use to separate the charge of TAT from the charge
of the SB2 peptide. The residue could be substituted by glycine.
TSBCTR:
RKKRRQRRRAALVLAVPLAHPTLA (SEQ ID NO:7) (control)

In one embodiment, the present invention is a therapeutic composition comprising an hsp90 decoy peptide. The therapeutic composition preferably comprises the hsp90 decoy peptide in combination with pharmaceutically acceptable carriers. Preferably, the peptide is in the form of pills, lozenges, formulations suitable for oral, i.v. or subcutaneous application and forms suitable for topical application.

Therapeutic Methods

In one embodiment, the present invention is a method for treating or reducing vascular diameter in a tissue of a human or non-human patient, for example in treatment of hypotension, septic shock, pulmonary edema, erythemalgia, or acne rosacea. Preferably one would administer 0.01-1 mg/kg per day of a hsp90 decoy peptide, preferably the TSB2 peptide (SEQ ID NO:6 RKKRRQRRR-AELVLEVPLEHPTLE), preferably by i.v., i.p. or subcutaneous administration to a human or non-human patient. One would then examine the patient for reduction in the diameter of the blood vessels in the treated tissue. Preferably, one would administer the daily dose at one time. One would understand that an effective amount of the compound had been administered when one sees a reduction of disease symptoms. To determine whether the diameter of the blood vessels has been reduced, one could preferably use pulse oximetry for blood flow or blood pressure measurements for shock symptoms.

Upon entering the blood stream, hsp90 decoy peptide will have complete access to the endothelium of vascular tissues. During hypotension due to septic shock, vascular tissues generate too much nitric oxide (.NO). From the blood stream TSB2 will enter the endothelium and via disruption of hsp90-dependent interactions with eNOS will convert eNOS from an .NO generating enzyme into a superoxide anion (O₂.⁻) generating NADPH oxygenase. The increased O₂.⁻ will scavenge the excess .NO and thereby decrease vasodilation and increase blood pressure.

Another embodiment of the present invention is a method for treating cancer, preferably by administering 0.01-1 mg/kg of a hsp90 decoy peptide, preferably the TSB2 peptide (SEQ ID NO:6 RKKRRQRRR-AELVLEVPLEHPTLE), preferably by oral, i.v., i.p. or subcutaneous administration to a human or non-human animal to reduce vascular diameter in a tumor or reduce the growth of new blood vessels by inhibiting the proliferation of endothelial cells. Preferably, one would dose the patient over a long period of time, from several months to several years. The preferable cancer would be vascular tumor cancers, such as melanoma and hemangiomas. One would understand that an effective amount of compound had been delivered when one saw reduction in disease symptoms, such as tumor shrinkage.

During EC proliferation, hsp90 association with eNOS is absolutely essential for maintaining a high level of .NO generation and cell division. TSB2 will enter the proliferating EC from the blood stream and disrupt hsp90-dependent interactions with eNOS. Loss of this chaperone-dependent interaction with eNOS, and possibly other chaperone dependent activities, will convert eNOS from an .NO generating enzyme into a superoxide anion (O₂.⁻) generating NADPH oxygenase. The increased O₂.⁻ will scavenge not only .NO required for proliferation and but also disrupt other hsp90-dependent interactions required for proliferation.

Another embodiment of the present invention is a method for treating cancer by administering 0.01-1 mg/kg of a hsp90 decoy peptide, preferably the TSB2 peptide by (SEQ ID NO:6 RKKRRQRRR-AELVLEVPLEHPTLE) via topical administration to a human or non-human animal to reduce vascularization and growth of hemangiomas. Preferably, the treatment progresses until the tumor diminishes or disappears. When TSB2 applied topically to the hemangioma, it will enter the vascularized tissue and disrupt hsp90-dependent chaperone activity thus increasing oxidative stress and apoptosis of the vascularized hemangiomas to promote cell death.

### EXAMPLES

Materials and Methods

Peptide Synthesis: Twelve overlapping peptides (B1-B12, 20 mers) were designed spanning the entire region of where hsp90 was reported to associate with eNOS (aa 290-300).⁵ TAT protein transduction domain (PTD)⁶, PEP1⁷ and eNOS-derived peptides were synthesized using Fmoc chemistry by Dr. Basam Wakim in the Protein, Nucleic Acid shared core facility at MCW and purified by HPLC. The peptides were HPLC purified and predicted molecular weights confirmed by MALDI-TOF mass spectrometry.

Proliferating Endothelial Cells (EC) and Disruption of hsp90 Interactions with eNOS in EC lysates: Bovine aortic endothelial cells were expanded and maintained in RPMI-1640 media containing 10% FBS, antibiotics and mycotics. BAEC cultures were passaged with trypsin-EDTA and used for experiments between passage 5-7.

Previous studies from this laboratory revealed that proliferating ECs had a much higher level of hsp90 association with eNOS than confluent, non-proliferating ECs.⁴ Accordingly, to identify an eNOS-derived peptide that could disrupt hsp90 interactions with eNOS we made cell lysates from proliferating BAEC cultures. Confluent BAEC cultures were passaged and allowed to proliferate as previously described.⁴ Proliferating BAEC in 100 mm dishes (10-20 dishes) were lysed in modified RIPA buffer (50 mM Tris HCI, pH 7.5, 1% NP40, 0.1 mM EDTA, 0.1 mM EGTA, 0.1% SDS, 0.1% deoxycholic acid, 1× protease inhibitors (Sigma), 1× phosphatase inhibitors (Sigma). Cell lysates were transferred to 1.5 mL microcentrifuge tubes, placed on ice, sonicated 2 times (30 sec) and cell debris isolated by centrifugation (14,000×g, 3 min, 4° C.).⁴ Supernatants were removed from the cell debris, pooled and cell protein determined in the supernatants using BCA reagent. An aliquot of the proliferating EC lysates (300 μg in 0.5 mL) was precleared with protein A/G (20 μL of a 50% slurry, 2 hours at 4° C.) and then incubated with each of the individual decoy peptides (5 μg, for a final concentration of 10 μg/mL) for 2 hours at 4° C. Next, eNOS was immunoprecipitated from the incubations using H32 antibody from BioMol (1 μg/100 μg of cell lysate) as previously described.⁴ ProteinA/G (50 μL of a 50% slurry) was added to isolate the immunoprecipitates. eNOS and its associated proteins were separated by SDS-PAGE (7.5% gel), transferred to nitrocellulose and immunoblotted for eNOS and hsp90 as previously described.⁴

Effects of eNOS-derived peptides on stimulated .NO production and hsp90 association with eNOS in BAEC cultures: BAEC were cultured and maintained in 100 mm culture dishes until confluent. The eNOS-derived peptide B2 was incubated for 30 min with PEP1, a protein transduction domain peptide⁷ (mole:mole=1:20) and then added to the BAEC cultures at a final peptide concentration of 5 nM. BAEC cultures were incubated with either nothing (control), PEP1 alone (transduction control) or B2+PEP1. These pretreated BAEC cultures were washed and incubated in HBSS containing L-arginine (25 μM) at 37° C. for 15 min to obtain basal .NO production or A23187 (5 μM)+L-arginine (25 μM), a calcium-ionophore to stimulate eNOS-dependent .NO production. The incubation buffers were removed and stored on ice for immediate nitrite+nitrate analysis by ozone chemiluminescence as described.⁸ BAEC proteins were lysed in 400 μL of MOPS lysis buffer (20 mM MOPS, pH 7.0, 2 mM EGTA, 5 mM EDTA, 30 mM sodium fluoride, 40 mM β-glycerophosphate, pH 7.2, 10 mM sodium pyrophosphate, 0.5% NP-40, 1× Protease inhibitors (Sigma), 1× Phosphatase Inhibitors (Sigma) and subjected to eNOS immunoprecipitation as described above. eNOS and the levels of associated hsp90 in the immunoprecipitates were determined by western blot analysis as described above.

Vasodilation Studies: Previous studies showed that the facialis artery of mice vasodilated by an eNOS-dependent mechanism.^{3, 4} To determine the effects of the B2 peptide on vasodilation, we redesigned the B2 peptide to contain a PTD. The B2 peptide was also shortened to 15 aa (AELVLEVPLEHPTLE) with the TAT PTD (RKKRRQRRR) added at the N-terminal as described by Pagano and associates.⁶ (Note that the initial A residue is a linker.) This redesign made a new decoy peptide (RKKRRQRRR-AELVLEVPLEHPTLE), which we called TSB2. In designing a control peptide, TSB(Ctr) (RKKRRQRRR-AALVLAVPLAHPRLA), we reasoned that if E were important for binding, then replacing E with A would result in a peptide that would fail to bind to hsp90 or another heatshock protein essential to chaperone-dependent signaling and possibly mitochondrial function and therefore fail to impair vasodilation. To test these ideas, TSB2 and TSB(Ctr) (10 μg/mL) were incubated with separatefacialis arteries from healthy C57BL/6 male mice, whose vasodilation is mediated 100% by eNOS.⁹ Ten minutes later, excess peptides were removed by changing the buffer and ACh-dose response curves of the preconstricted and pressurized vessels determined as before.⁹

To determine if TSB2 effectively inhibited EC and eNOS-dependent vasodilation in mice, we injected C57BL/6 mice with TSB2 (1 mg/kg/day) for 2 weeks. Facialis arteries were isolated, hung in organ vessel chambers, pressurized to 60 mmHg and examined for changes in ACh-dependent vasodilation as described above.³

Effects of TSB2 on EC- and eNOS-dependent O₂.⁻ generation in situ: To determine if TSB2 altered O₂.⁻ generation in native EC on vascular tissues, anesthetized C57BL/6 mice were sacrificed by esanguination and their aortas perfused at a rate of 2 mL/min with MOPS buffer containing TSB2 (10 μg/mL) and hydroethidine (10 μM) in the absence and presence of L-NAME (200 μM) for 10 minutes followed by washout of excess, unreacted hydroethidine with MOPS buffer alone. Hydroethidine is a cell permeable probe that upon reaction with O₂.⁻ is converted to a fluorescent 2-OH-ethidine⁺ product that can be quantified by fluorescent microscopy or HPLC.^{2, 10, 11} Aortas were quickly excised and examined for nuclear 2-OH-ethidine⁺ staining (an index of O₂.⁻ generation) using confocal fluorescent microscopy as previously described.^{2, 10}

Effects of TSB2 on the proliferation of EC and K562 cells: Endothelial cells (BAEC) were seeded at 2.5×10⁶ per 100 mm dish and allowed to grow in RPMI1640 with 10% FBS. Starting from the second day, cells were fed everyday with fresh growth medium together with and without 100 mg/ml TSB2. On the fourth day, cells were trypsinized and cell number determined by hemacytometer. In parallel experiments, leukemia cells (K562) were seeded at 1×10⁶ per 100 mm dish and allowed to grow in serum free RPMI1640 with and without 10 mg/ml TSB2. On the second day, cell number was determined using hemocytometer.

Effects of TSB2 on growth of melanoma tumors in C57BL/6 mice: Fifteen C57BL/6 mice were injected with 2×10⁵ B16 melanoma tumor cells per mice subcutaneously on the back. Ten mice were injected intraperitoneal (i.p.) injections daily with 100 μl of TSB2 peptide at a dose of 1 mg/kg (5 mice) and 3 mg/kg (5 mice). Tumor volumes from all fifteen mice were measured starting from 12 days post-transplantation. All mice were sacrificed on the 17th day post-transplantation. Tumors were removed by microdissection, weighed and fixed in 10% formalin for histology studies at a later date.

Effects of TSB2 on the Generation of Angiostatic Factors in vivo: To determine if TSB2 increases the production of angiostatic factors in mice, the hearts from the C57BL/6 mice that were treated with TSB2 (1 mg/kg, 2 weeks, see above) were examined by immunofluorescence using a specific antibody to detect angiostatin. Hearts were removed, flash frozen in OCT. Sections of the hearts were cut and then examined by immunofluorescence for the presence of angiostatin using previously established protocols.

Results

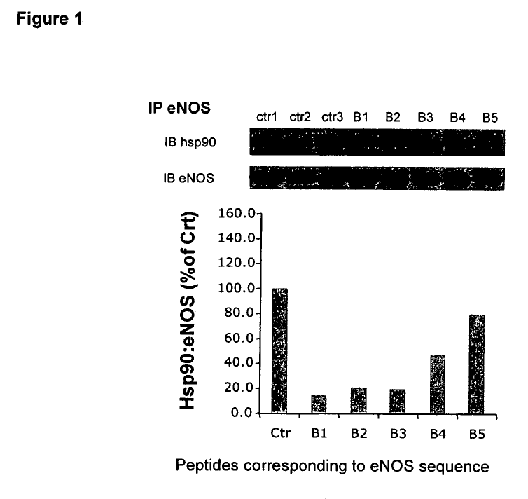

Effects of eNOS-derived peptides on hsp90 association with eNOS: The 12 eNOS-derived peptides were incubated individually with lysates of proliferating BAEC cultures before immunoprecipitation of eNOS. The immunoprecipitated eNOS and associated proteins were analyzed by western blot analysis for hsp90 and eNOS. Out of the 12 peptides only B1-B5 inhibited hsp90-eNOS interactions, and within this group B1 (291-310aa), B2 (301-320aa), and B3 (311-330aa) significantly impaired association (FIG. 1, ˜80%, p<0.05, n=5).

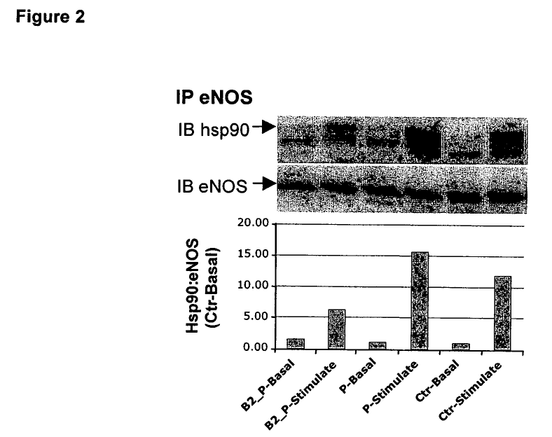

Effects of B2 on basal and stimulated eNOS-dependent .NO generation and hsp90 association with eNOS: The B2:PEP1 mixture significantly inhibited basal and A23187 stimulated .NO production compared to BAEC cultures treated with PEP1 alone or nothing (control). Immunoprecipitates of eNOS from these test groups reveal the B2:PEP1 mixture markedly decreased hsp90 association not only under basal conditions but also when the cultures were stimulated with A23187. Under these conditions TSB2 decreased hsp90 association to ¼^{th} of the levels in BAEC cultures treated with PEP1 alone or nothing (FIG. 2, p<0.05, n=3).

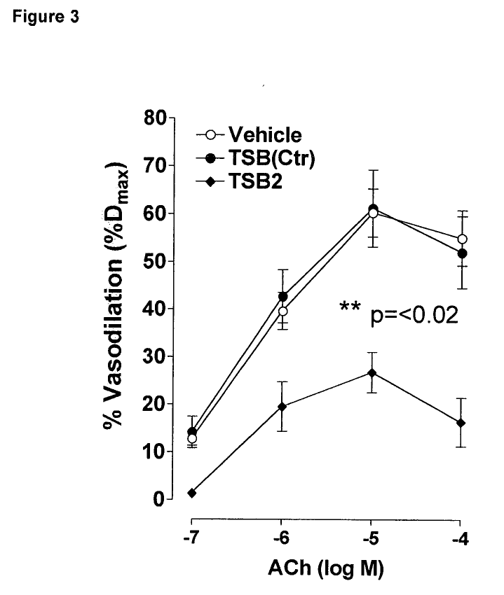

Effects of TSB2 on EC- and eNOS-dependent vasodilation: Pre-treating pressurized facialis arteries with TSB2 (n=7) significantly decreased ACh-induced vasodilation by >50% while the modified TSB(Ctr) (Es replaced with A) had no effect on vasodilation (n=6) compared to vehicle control (n=10) (FIG. 3, p<0.02).

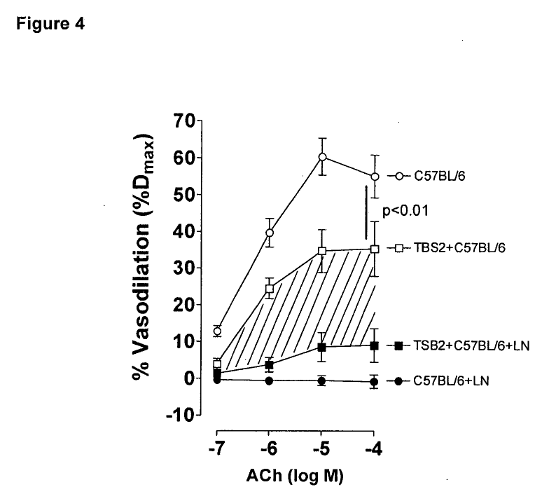

Effects of TSB2 on EC- and eNOS-dependent Vasodilation Ex Vivo: Next we reasoned that if hsp90 association with eNOS is important for vascular function, then chronic treatments with TSB2 should inhibit EC- and eNOS-dependent vasodilation in the C57BL/6 mice. To test this hypothesis, we treated C57BL/6 mice with TSB2 (1 mg/kg/day) or PBS for 2 weeks. After 2 weeks, the C57BL/6 mice were anesthetized, sacrificed by exsanguination and facialis arteries isolated and pressurized as before.⁹ 12 During isolation we noted that the facialis arteries from control C57BL/6 mice had thin layers of connective tissue on the advential side of the vessel. When facialis arteries from TSB2-treated mice were examined, connective tissue on the surface of the vessel appeared as large, thick fibers crisscrossing the adventia (data not shown). FIG. 4 shows chronic treatments of C57BL/6 mice with TSB2 reduces eNOS-dependent vasodilation of facialis arteries (hatched area) in C57BL/6 mice compared to vasodilation of control vessels from control C57BL/6 mice.

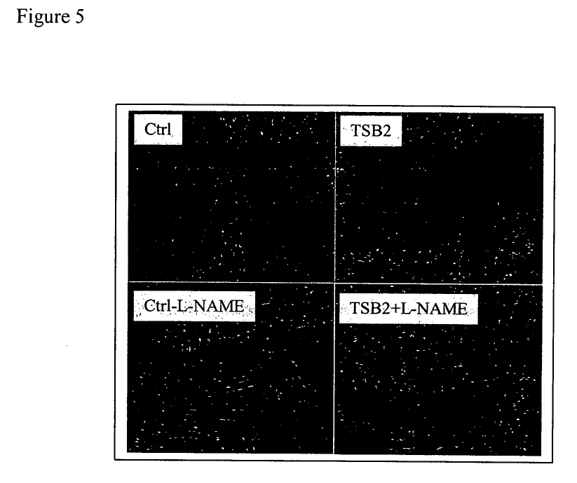

Effects of TSB2 on eNOS-dependent O₂.⁻ Generation in Native EC: To test the hypothesis that disruption of hsp90 interactions with eNOS uncouples eNOS activity in vascular EC, we perfused aortas of mice in situ with TSB2 and hydroethidine under a physiological flow rate of 2 mL/min and then rapidly removed the vessels and analyzed the fluorescent intensity in the native EC by fluorescent confocal microscopy. Images in FIG. 5 show that perfusion with TSB2 markedly increases hydroethidine staining, an index of O₂.⁻ generation (upper right compared to upper left) by mechanism that could be inhibited in part by L-NAME (lower right). On the basis that L-NAME is a substrate-specific inhibitor that blocks both .NO and O₂.⁻ generation from eNOS¹⁸, these data confirm that acute exposure to TSB2 uncouples eNOS activity to increase eNOS-dependent O₂.⁻ generation in native EC on isolated aortas.

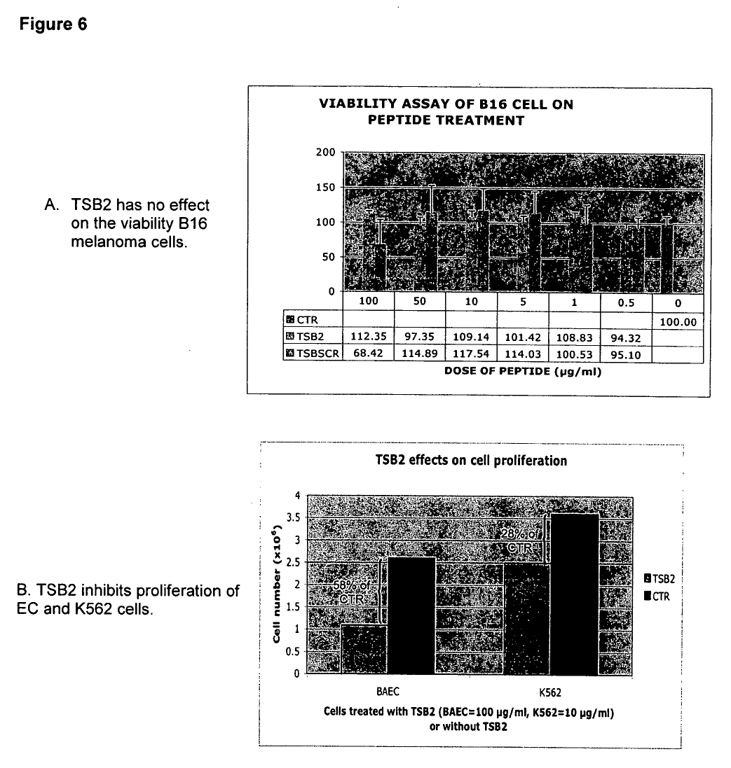

Effects of TSB2 on the proliferation of EC and K562 cells: To determine if TSB2 had any direct inhibitory effects on proliferation of cells, we added TSB2 (10 μg/mL, final concentration) to the culture media of B16 melanoma cells, EC and K562 cells, a leukemia cell-line and then quantified changes in cell number compared to untreated cultures. TSB2 had no direct effect on the viability of B16 melanoma cells in culture, based on a lack of change in LDH release, when used even at 100 μg/mL. However, TSB2 did inhibit EC proliferation by 58% of controls when used at 100 μg/mL and inhibited proliferation of K562 cells by 28% of controls when used at 10 μg/mL (FIG. 6). These data demonstrate that TSB2 has little direct cytotoxic effects on B16 melanoma cells in culture but can inhibit proliferation of EC and a leukemia cancer cell-line. When these data are interpreted in the context of TSB2 effects on B16 melanoma tumors in the mice, it suggests that TSB2 works better in vivo than in vitro (see below). Possibly, TSB2 is inducing mechanisms of anti-angiogenesis other than disrupting hsp90 interactions with eNOS.

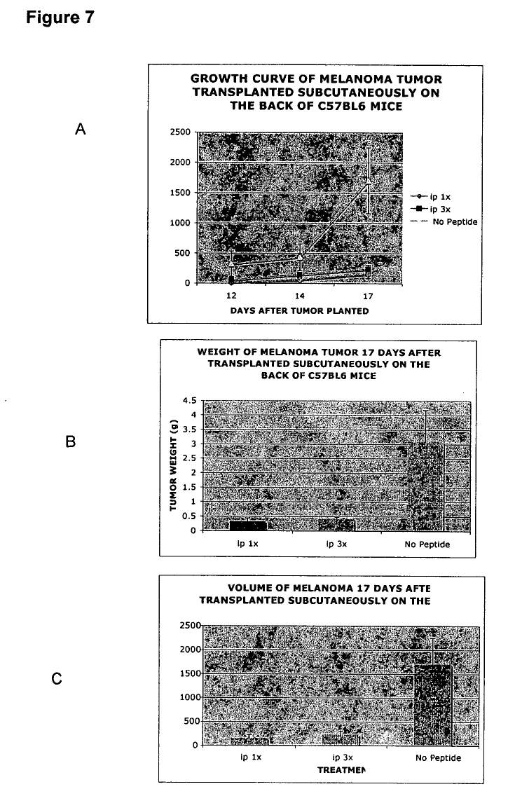

Effects of TSB2 on the growth of B16 melanoma tumors in C57BL/6 mice: To determine in vivo effects of TSB2 on the growth of a solid tumor, we injected C57BL/6 mice with B16 melanoma tumor cells and then treated the mice with TSB2 at a 1× dose (1 mg/kg) per day and a 3× dose (3 mg/kg) per day for 17 days. Volumes of the tumors were measured and plotted to obtain growth curves. At the end of the study, the tumors were removed, weighed, volume measured and placed in 10% formalin for preservation. The growth of melanoma tumors in either the 1× or 3×TSB2 treated mice were markedly reduced compared to the growth in untreated mice (FIG. 7A). At the end of the 17 day period, the tumors in the mice were removed by dissection and assessed for weight and volume. TSB2 treatments, both 1× and 3× doses, significantly decreased the final weight and volume of the tumors compared to tumors in untreated C57BL/6 mice (FIGS. 7B and 7C, respectively).

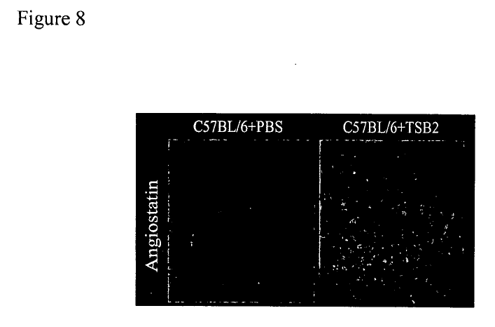

Effects of TSB2 on myocardial angiostatin production in vivo: To determine if TSB2 increased production of angiostatic factors in mice, hearts from C57BL/6 mice that were treated with TSB2 (1 mg/kg, 2 weeks) were examined by immunofluorescence using a specific antibody that detects angiostatin. FIG. 8 shows that chronic treatment of C57BL/6 mice increases the production of angiostatin in the myocardium of C57BL/6 mice compared to untreated mice. Thus, TSB2 increases the generation of angiostatin in vascular tissues, which, can in turn, inhibit angiogenesis. Such mechanisms may be less prominent in vitro than in vivo because of the lack of plasminogen in culture.

Discussion

Studies here describe the development of a novel eNOS-derived decoy peptide that can be used to impair eNOS-dependent vascular function including growth of melanoma tumors. We have shown that small peptides derived from eNOS can be used to disrupt chaperone-dependent signaling with eNOS to inhibit eNOS-dependent .NO generation and vasodilation by a mechanism that actually changes this enzyme's function to increase O₂.⁻ generation rather than .NO. As .NO plays in an important role in tumor angiogenesis we reasoned that the redesigned TSB2 decoy peptide may be an effective inhibitor of tumor angiogenesis. Our studies show that while TSB2 has little cytotoxic effects on B16 melanoma cells in culture, it is highly effective at inhibiting the growth of tumors in C57BL/6 mice. Interestingly, TSB2 can also inhibit proliferation of EC and K562 cells. Exactly how TSB2 blocks the growth of the melanoma tumor in vivo is unclear. However, our studies suggest that one potential mechanism is TSB2 may increase the production of angiostatic factors in vascular tissues as a means of enhancing its anti-angiogenic effects.

When we designed TSB2 we included TAT, a protein transduction domain that was proven to increase translocation of other small decoy peptides for inhibiting NADPH oxidoreductase activity in vascular tissues.⁶ TAT facilitates translocation of peptides or proteins through all cells. As the endothelium of tumors is believed to express proteins and receptors that are distinct from the proteins and receptors of normal vascular tissue, it may be possible to exchange TAT for a protein, antibody or receptor that specifically targets the endothelium of tumors i.e., VEGFreceptor.^{13, 14} Thus, adding other functional groups to the SB2 peptide (or enclosing SB2 or TSB2 in a stealth liposome that has targeting domains on its surface) may markedly increase the SB2's ability to inhibit angiogenesis in tumors without adversely effecting vascular function in healthy tissue.

As TSB2 induces eNOS to switch from generating .NO to O₂.⁻, a free radical whose physiological effects are diametrically opposed to those of .NO, TSB2 may be an important therapeutic agent for other disease states. For example, recent reports show VEGF-stimulated .NO production appears to drive hypotension and shock during sepsis. ^{15, 16} Thus, TSB2 treatments could increase vascular O₂.⁻ generation to off-set the increased production of .NO that occurs in sepsis.

Our studies identified the specific binding site on eNOS where hsp90 associates with eNOS (301-320aa). Here we showed that B1, B2 and B3 are all capable of disrupting hsp90 association with eNOS in cell lysates. Cell lysates from proliferating BAEC were used to screen the eNOS-derived peptides for two important reasons. First, proliferating BAEC are known to possess a high level of hsp90 association with eNOS that is essential for maintaining a high level of BAEC .NO generation and EC proliferation.³ Second, conducting the studies with lysates from proliferating BAEC rather than intact EC cultures, removes confounding variables of cell physiology and metabolism. Thus, findings from these studies are justifiably restricted to protein-protein interactions.

After determining where hsp90 bound to eNOS, we reasoned that it might be advantageous to reduce the size of the B2 peptide and include a PTD to improve cellular uptake. Thus we redesigned B2 into TSB2. Using TSB2 we observed that this redesigned decoy peptide blocked hsp90 association with eNOS, inhibited stimulated .NO generation and inhibited vasodilation not only acutely in vitro (i.e., isolated vessels) but also after chronic treatments ex vivo (i.e., isolated vessels from a treated mouse). Taken together these findings indicate that 1) B2 and its derivatives can be used to disrupt chaperone-dependent protein-protein interactions with eNOS; 2) they can be used to delineate the cellular mechanisms by which chaperones mediate eNOS function as it relates to free radial product formation; and, 3) they can be used to investigate mechanisms of vasodilation even in vivo. Findings from our studies may have importance for understanding the role of other chaperone proteins and how their signaling and/or interactions with eNOS may influence vascular function.

When we compare our observations that TSB2 dramatically decreased the growth of melanoma tumors by 75% to the observations that TSB2 had no direct effect on the viability of B16 melanoma cells in culture but did decrease proliferation of EC and K562 leukemia cells, it suggests TSB2 works better in vivo than in vitro. Exactly how TSB2 is achieving such a dramatic effect in vivo remains unclear. However, based on the fact that inhibition of vascular .NO production with L-NAME increases generation of angiostatin¹⁷ and TSB2 disrupts hsp90 interaction with eNOS to uncouple enzyme activity, allowing it to generate O₂.⁻, it is possible TSB2 increases the production of angiostatin in vivo. To determine if TSB2 increases angiostatin in vivo, we examined the hearts of the C57BL/6 mice that were treated for 2 weeks with TSB2. Immunofluorescence studies revealed that the hearts of these mice generate high levels of angiostatin. Taken together these data indicate that the SB2 peptide disrupts usual eNOS-related chaperone-dependent activity in such a way that the affected tissue begin to generate angiostatic factors. Accordingly, future studies should be aimed at identifying proteins, peptides or novel delivery systems that are able to direct the SB2 peptide to tumors so that its anti-angiogenic effects may be better localized to the tumor thereby minimizing potential adverse effects in healthy tissue. Alternatively, expression systems could be developed to target the gene for SB2 and potential variants in tumors to ensure local synthesis of the SB2 peptide or the TSB2 peptide in the tumor itself.

### REFERENCES

1. Garcia-Cardena G, Fan R, Shah V, Sorrentino R, Cirino G, Papapetropoulos A, Sessa W C: Dynamic activation of endothelial nitric oxide synthase by Hsp90, Nature 1998, 392:821-824
2. Pritchard K A, Jr., Ackerman A W, Gross E R, Stepp D W, Shi Y, Fontana J T, Baker J E, Sessa W C: Heat shock protein 90 mediates the balance of nitric oxide and superoxide anion from endothelial nitric-oxide synthase, J Biol Chem 2001, 276:17621-17624
3. Ou J, Fontana J T, Ou Z, Jones D W, Ackerman A W, Oldham K T, Yu J, Sessa W C, Pritchard K A, Jr.: Heat shock protein 90 and tyrosine kinase regulate eNOS .NO generation but not .NO bioactivity, Am J Physiol Heart Circ Physiol 2004, 286:H561-H569
4. Ou J, Ou Z, Ackerman A W, Oldham K T, Pritchard K A, Jr.: Inhibition of heat shock protein 90 (hsp90) in proliferating endothelial cells uncouples endothelial nitric oxide synthase activity, Free Radic Biol Med 2003, 34:269-276
5. Fontana J, Fulton D, Chen Y, Fairchild T A, McCabe T J, Fujita N, Tsuruo T, Sessa W C: Domain mapping studies reveal that the M domain of hsp90 serves as a molecular scaffold to regulate Akt-dependent phosphorylation of endothelial nitric oxide synthase and NO release, Circ Res 2002, 90:866-873
6. Rey F E, Cifuentes M E, Kiarash A, Quinn M T, Pagano P J: Novel Competitive Inhibitor of NAD(P)H Oxidase Assembly Attenuates Vascular O2- and Systolic Blood Pressure in Mice, Circ Res 2001, 89:408-414
7. Morris M C, Depollier J, Mery J, Heitz F, Divita G: A peptide carrier for the delivery of biologically active proteins into mammalian cells, Nat Biotechnol 2001, 19:1173-1176
8. Ou Z, Ou J, Ackerman A W, Oldham K T, Pritchard K A, Jr.: L-4F, an apolipoprotein A-1 mimetic, restores nitric oxide and superoxide anion balance in low-density lipoprotein-treated endothelial cells, Circulation 2003, 107:1520-1524
9. Ou J, Ou Z, Jones D W, Holzhauer S, Hatoum O A, Ackerman A W, Weihrauch D W, Gutterman D D, Guice K, Oldham K T, Hillery C A, Pritchard K A, Jr.: L-4F, an apolipoprotein A-1 mimetic, dramatically improves vasodilation in hypercholesterolemia and sickle cell disease, Circulation 2003, 107:2337-2341
10. Stepp D W, Ou J, Ackerman A W, Welak S, Klick D, Pritchard Jr. K A: Native LDL and minimally oxidized LDL differentially regulate superoxide anion in vascular endothelium In Situ, Am. J. Physiology Heart and Circulation 2002, 283:H750-H759
11. Zhao H, Kalivendi S, Zhang H, Joseph J, Nithipatikom K, Vasquez-Vivar J, Kalyanaraman B: Superoxide reacts with hydroethidine but forms a fluorescent product that is distinctly different from ethidium: potential implications in intracellular fluorescence detection of superoxide, Free Radic Biol Med 2003, 34:1359-1368
12. Ou J, Wang J, Xu H, Ou Z, Sorci-Thomas M G, Jones D W, Signorino P, Densmore J C, Kaul S, Oldham K T, Pritchard K A, Jr.: Effects of D-4F on vasodilation and vessel wall thickness in hypercholesterolemic LDL receptor-null and LDL receptor/apolipoprotein A-I double-knockout mice on Western diet, Circ Res 2005, 97:1190-1197
13. Inoue K, Slaton J W, Davis D W, Hicklin D J, McConkey D J, Karashima T, Radinsky R, Dinney C P: Treatment of human metastatic transitional cell carcinoma of the bladder in a murine model with the anti-vascular endothelial growth factor receptor monoclonal antibody DC101 and paclitaxel, Clin Cancer Res 2000, 6:2635-2643
14. Folpe A L, Veikkola T, Valtola R, Weiss S W: Vascular endothelial growth factor receptor-3 (VEGFR-3): a marker of vascular tumors with presumed lymphatic differentiation, including Kaposi's sarcoma, kaposiform and Dabska-type hemangioendotheliomas, and a subset of angiosarcomas, Mod Pathol 2000,13:180-185
15. Pickkers P, Sprong T, Eijk L, Hoeven H, Smits P, Deuren M: Vascular endothelial growth factor is increased during the first 48 hours of human septic shock and correlates with vascular permeability, Shock 2005, 24:508-512
16. Yano K, Liaw P C, Mullington J M, Shih S C, Okada H, Bodyak N, Kang P M, Toltl L, Belikoff B, Buras J, Simms B T, Mizgerd J P, Carmeliet P, Karumanchi S A, Aird W C: Vascular endothelial growth factor is an important determinant of sepsis morbidity and mortality, J Exp Med 2006, 203:1447-1458
17. Matsunaga T, Weihrauch D W, Moniz M C, Tessmer J, Warltier D C, Chilian W M: Angiostatin inhibits coronary angiogenesis during impaired production of nitric oxide, Circulation 2002,105:2185-2191
18. Vasquez-Vivar, et al., Proc. Natl. Acad. Sci., 95:9220-25 (1998)

We claim:

1. A pharmaceutical preparation of an hsp90 decoy peptide, wherein the peptide comprises SB2 (SEQ ID NO:5).

2. The peptide of claim 1 additionally comprising a protein translocation domain.

3. The peptide of claim 2 wherein the translocation domain is TAT.

4. The peptide of claim 2 wherein the peptide is identical to TSB2 (SEQ ID NO:6).

5. A pharmaceutical preparation of an hsp90 decoy peptide, wherein the peptide is B1 (SEQ ID NO:2), B2 (SEQ ID NO:3) or B3 (SEQ ID NO:4).

6. The pharmaceutical composition of claim 5, wherein the peptide additionally comprises a protein translocation domain.

7. A method of inhibiting hsp90 association with eNOS in a patient, comprising the step of treating a patient with an effective amount of the pharmaceutical composition comprising an hsp90 decoy peptide.

8. The method of claim 7 wherein the pharmaceutical peptide comprises a protein translocation domain.

9. The method of claim 7 wherein the patient is suffering from hypotension, septic shock, pulmonary adema, erythromalgia or acne rosacea.

10. The method of claim 8 wherein the patient is suffering from hypotension, septic shock, pulmonary adema, erythromalgia or acne rosacea.

11. The method of claim 7 wherein the patient is a tumor patient.

12. The method of claim 7 wherein the patient has a disease or condition associated with abnormal, excessive blood vessel development.

13. The method of claim 7 wherein the patient is a hemangioma patient.

14. The method of claim 7 wherein the peptide compsrises peptide SB2.

15. The method of claim 14 wherein the petide is TSB2 (SEQ ID NO:6).

16. The method of claim 7 wherein the peptide is selected from the group consisting of B1 (SEQ ID NO:2), B2 (SEQ ID NO:3) and B3 (SEQ ID NO:4).

17. The method of claim 8 wherein the protein translocation domain is linked to the hsp90 decoy peptide with an amino acid residue.

18. The method of claim 17 wherein TSB2 (SEQ ID NO:6) is linked to the TAT translocation domain.


SEQUENCE LISTING (12 sequences)

SEQ ID NO: 1 - protein, 40 aa, Artificial
  Synthetic polypeptide
LPLLLQAPDEAPELFVLPPELVLEVPLEHPTLEWFAALGL

SEQ ID NO: 2 - protein, 20 aa, Artificial
  Synthetic polypeptide
LPLLLQAPDEAPELFVLPPE

SEQ ID NO: 3 - protein, 20 aa, Artificial
  Synthetic polypeptide
APELFVLPPELVLEVPLEHP

SEQ ID NO: 4 - protein, 20 aa, Artificial
  Synthetic polypeptide
LVLEVPLEHPTLEWFAALGL

SEQ ID NO: 5 - protein, 14 aa, Artificial
  Synthetic polypeptide
ELVLEVPLEHPTLE

SEQ ID NO: 6 - protein, 24 aa, Artificial
  Synthetic polypeptide
RKKRRQRRRAELVLEVPLEHPTLE

SEQ ID NO: 7 - protein, 24 aa, Artificial
  Synthetic polypeptide
RKKRRQRRRAALVLAVPLAHPTLA

SEQ ID NO: 8 - protein, 40 aa, Artificial
  Synthetic polypeptide
LPLLLQAPDDPPELFLLPPELVLEVPLEHPTLEWFAALGL

SEQ ID NO: 9 - protein, 21 aa, Artificial
  Synthetic polypeptide
KETWWETWWTEWSQPKKKRKV

SEQ ID NO: 10 - protein, 15 aa, Artificial
  Synthetic polypeptide
AELVLEVPLEHPTLE

SEQ ID NO: 11 - protein, 9 aa, Artificial
  Synthetic polypeptide
RKKRRQRRR

SEQ ID NO: 12 - protein, 24 aa, Artificial
  Synthetic polypeptide
RKKRRQRRRAALVLAVPLAHPRLA